(19) United States
(12) Patent Application Publication
(10) Pub. No.: US 2022/0000796 A1
(43) Pub. Date: Jan. 6, 2022

(54) COMPOSITIONS COMPRISING CBD FOR TREATING ANORECTAL DISORDERS

(71) Applicant: UNV MEDICINE LTD., Ashkelon (IL)

(72) Inventors: Omer LAYANI, Ashkelon (IL); Ilana LAVON, Ashkelon (IL); Shaul MUKHTAR, Ashkelon (IL)

(21) Appl. No.: 17/281,670

(22) PCT Filed: Sep. 26, 2019
(86) PCT No.: PCT/IL2019/051064

§ 371 Date: Mar. 31, 2021

(30) Foreign Application Priority Data
Oct. 2, 2018 (IL) 262050

(51) Int. Cl. A61K 31/05; A61K 36/63; A61K 36/8965; A61K 36/287; A61P 29/00; A61K 9/00

(52) U.S. Cl. CPC A61K 31/05; A61K 36/63; A61K 9/0014; A61K 36/287; A61P 29/00; A61K 36/8965

(57) ABSTRACT

The present invention discloses compositions comprising CBD and several herbal extracts and powders, and methods for the treatment of anorectal disorders such as hemorrhoids. In particular, the invention provides compositions useful in the treatment and alleviation of hemorrhoids and related symptoms

### FIELD OF THE INVENTION

The present invention relates to compositions and methods for the treatment and management of anorectal disorder such as hemorrhoids.

### BACKGROUND OF THE INVENTION

Anorectal disorders are a group of medical disorders that occur at the junction of the anal canal and the rectum. The most common anorectal disorders include hemorrhoids, anal fissures, fecal incontinence, proctalgia fugax, and excessive perineal descent. Hemorrhoids, also called piles, are vascular structures in the anal canal. In their normal state, they are cushions that help with stool control. They become an anorectal disease when swollen or inflamed; and the term “hemorrhoid” is often used to refer to the disease. Hemorrhoids can be internal or external. Internal hemorrhoids often result in painless, bright red rectal bleeding when defecating. External hemorrhoids often result in pain and swelling in the area of the anus.

Various topical compositions and suppositories are available for the treatment of hemorrhoids, however, little evidence supports their beneficial use. Steroid-containing agents should not be used for more than 14 days as they may cause thinning of the skin. Most compositions include a combination of active ingredients. These may include a barrier cream such as petroleum jelly or zinc oxide, an analgesic agent such as lidocaine, and a vasoconstrictor such as epinephrine, however, the efficacy of such compositions has not been proven.

The cannabis plant has long been used for medicinal and recreational purposes alike. Subsequently, this plant has been characterized as containing many naturally occurring substances of great interest in the fields of science and medicine. The medical use of cannabis is now legal in many countries under specific “medical cannabis” legislation. Medical cannabis is used for treating and alleviating symptoms associated with a growing number of indications, including pain, anorexia, asthma, glaucoma, arthritis, spasms, anxiety, and substance withdrawal. Many other illnesses are emerging as potential cannabis-responsive indications.

There remains a need for safe and efficient compositions for the efficient treatment and management of hemorrhoids, by providing an enhanced relief with little or no side effects.

### SUMMARY OF THE INVENTION

The present invention in embodiments thereof relates to compositions and methods for the treatment of anorectal disorder such as hemorrhoids, wherein the compositions include cannabis extract. In particular, embodiments of the invention provide compositions useful in the treatment of hemorrhoids and amelioration of symptoms associated therewith.

The invention is based, in part on the development of formulations (compositions), having unexpectedly advantageous properties in the treatment of hemorrhoids. In various embodiments, the compositions were found to exhibit marked and consistent efficacy and safety in the treatment of hemorrhoids, and/or symptoms associated therewith, while being efficient and having reduced side effects. In various embodiments, the compositions were found to be particularly useful as localized, topical medicaments. According to other embodiments, the compositions were found to exhibit enhanced absorption, tolerability, and efficiency, as disclosed herein.

In particular, it was discovered that compositions comprising oil extracts from cannabidiol (CBD)-rich (CBD-dominant) cannabis strains were more effective in alleviating hemorrhoids than compositions comprising tetrahydrocannabinol (THC)-dominant strains.

The compositions of the invention comprise a unique combination of ingredients from cannabis and other botanical sources. This unique combination provides advantageous properties over the use of each ingredient alone, which may be in various embodiments additive or greater than additive (synergistic), depending on the particular measured outcome.

According to the teaching of the invention, the composition comprises extracts obtained from cannabis (Cannabis Sativa L., e.g. Cannabis sativa and/or Cannabis indica strains approved for medical use).

According to one aspect, the present invention provides a composition comprising from about 1 to about 40% by weight of CBD oil, from about 5 to about 40% by weight of Jasminum flower extract, from about 5 to about 60% by weight of Pistacia lentiscus extract, from about 1 to about 35% by weight pomegranate peel powder and from about 1 to 35% by weight of Calendula flower extract or witch hazel extract.

According to a typical embodiment of the invention, the composition comprises CBD oil, namely a full-plant cannabis oil extraction, typically from cannabidiol (CBD)-dominant strains. An exemplary CBD-dominant cannabis strain comprises about 6 to about 30%, typically 10 to about 20% CBD, e.g. about 18% CBD, and up to 5%, typically 0.5 to about 3%, more typically 1 to about 2% each of tetrahydrocannabinol (THC) and cannabinol (CBN). For example, medical grade CBD oil as provided by the Israel Ministry of Health contains about 10 to about 30% CBD, in particular an average of 10%, 15%, 20% or 24% CBD, depending on the strain (wherein the initial extract may be diluted e.g. in olive oil or other pharmaceutically acceptable oils to obtain the determined concentration).

According to some embodiments, the CBD oil comprises from 30 to 85 wt %, from 35 to 80 wt %, from 40 to 75 wt %, from 45 to 70 wt %, from 50 to 65 wt %, from 45 to 80 wt %, or from 55 to 85 wt % of CBD.

According to some embodiments, the composition of the invention may include from about 1 to about 35% CBD oil by weight. According to some embodiments, the composition of the invention may include from about 2 to about 30% CBD oil by weight. According to some embodiments, the composition of the invention may include from about 4 to about 25% or from about 5 to about 20% CBD oil by weight. According to some embodiments, the composition of the invention may include from about 7.5 to about 15% CBD oil by weight.

According to some embodiments, the composition of the invention may include from about 10 to about 14% or to about 15% CBD oil by weight. According to some embodiments, the composition of the invention may include from about 11 to about 13% CBD oil by weight. In some exemplary embodiments, the composition of the invention may include about 12% CBD oil by weight. In some embodiment, the content of CBD oil in the composition may be replaced by statement of CBD in the composition. Thus, in some embodiments, the composition comprises from about 0.5 to about 33% by weight of CBD, from about 5 to about 40% by weight of Jasminum flower extract, from about 5 to about 60% by weight of Pistacia lentiscus extract, and from about 1 to about 35% by weight pomegranate peel powder, and about 1 to 25% by weight of witch hazel extract. According to other embodiments, the composition comprises from about 0.5 to about 33% by weight of CBD, from about 5 to about 40% by weight of Jasminum flower extract, from about 5 to about 60% by weight of Pistacia lentiscus extract, and from about 1 to about 35% by weight pomegranate peel powder, and about 1 to 25% by weight of Calendula flower extract. According to some embodiments, the composition comprises from 1 to 30 wt % or 2 to 25 wt % of CBD.

In some embodiments, one or more cannabinoids may be added to the composition in purified form. For example, CBD-containing oil may be manufactured synthetically, by addition of CBD (and optionally other cannabinoids and/or cannabis-derived compounds such as terpenes) to oil, which may subsequently be used in manufacturing the composition.

According to embodiments of the invention, the composition may further include pomegranate peel powder. Typically, pomegranate peel powder may be obtained from the peels of pomegranates by drying the peels and grinding them to a fine powder. According to some embodiments of the invention, the composition may include from about 1 to about 30%, from about 2.5 to about 25%, about 5 to about 20%, or about 7.5 to about 15%, e.g. about 10% by weight pomegranate peel powder.

According to embodiments of the invention, the composition may further include Jasminum flower extract. Typically, Jasminum flower extract may be obtained/extracted from the flowers of Jasminum. According to some embodiments of the invention, the composition may include from about 5 to about 40%, about 10 to about 30%, or about 15 to about 20%, e.g. about 17% by weight Jasminum flower extract.

According to embodiments of the invention, the composition may further include Pistacia lentiscus extract. Typically, Pistacia lentiscus extract may be obtained/extracted from the resin/leaves/fruits. According to some embodiments of the invention, the composition may include from about 5 to about 60%, about 10 to about 50%, or about 15 to about 40%, e.g. about 30% by weight Pistacia lentiscus extract.

According to embodiments of the invention, the composition may further include witch hazel extract. Typically, witch hazel extract may be obtained/extracted from the leaves and bark by ethanol extraction. According to some embodiments of the invention, the composition may include from about 2.5 to about 25%, from about 5 to about 20%, or from about 7.5 to about 15%, e.g. about 10% by weight witch hazel extract.

According to embodiments of the invention, the composition may further optionally include Calendula flower extract. Typically, Calendula flower extract may be obtained/extracted from the flowers. According to some embodiments of the invention, the composition may include from about 1 to about 30%, from about 2.5 to about 25%, about 5 to about 20%, or about 7.5 to about 15%, e.g. about 10% by weight Calendula flower extract.

According to embodiments of the invention, the composition may further optionally include Achillea millefolium extract. Typically, Achillea millefolium extract may be obtained/extracted from the leaves and flowers by ethanol extraction. According to some embodiments of the invention, the composition may include from about 1 to about 30%, from about 2.5 to about 25%, about 4 to about 20%, or about 6 to about 15%, e.g. about 7% by weight Achillea millefolium.

According to embodiments of the invention, the composition may further optionally include Ruscus aculeatus root extract. Typically, Ruscus aculeatus root extract may be obtained/extracted from the root and flowers, by ethanol extraction. According to some embodiments of the invention, the composition may include from about 1 to about 30%, from about 2.5 to about 25%, from about 4 to about 20%, or from about 6 to about 15%, e.g. about 7% by weight Ruscus aculeatus root extract.

According to some embodiments, the composition may further optionally include from 10 to about 95 wt % Coca butter.

According to some embodiments, there is thus provided a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight pomegranate peel powder, and from about 2.5 to about 15% by weight of witch hazel extract. According to another embodiment, the composition comprises from about 5 to about 30% by weight of CBD oil, from about 15 to about 30% by weight of Jasminum flower extract, from about 15 to about 50% by weight of Pistacia lentiscus extract, from about 5 to about 25% by weight pomegranate peel powder, from about 2.5 to about 15% by weight of witch hazel extract, from about 2.5 to about 25% by weight Calendula flower extract, from about 2.5 to about 25% by weight Achillea millefolium extract, and from about 2.5 to about 15% by weight Ruscus aculeatus root extract.

According to some embodiments, the composition may include about 8 to about 16% by weight of CBD oil. In some embodiments, the composition may include about 12% by weight of CBD oil. According to some embodiments, the CBD oil may include about 10 to about 30% by weight of CBD. According to other embodiments, the CBD oil may include about 40 to about 80% or from 55 to 80% by weight of CBD. According to some embodiments, the CBD oil may be obtained from plant extract, or may be prepared synthetically.

In some embodiments, the composition may include about 10% by weight of pomegranate peel powder. In some embodiments, the composition may include about 17% by weight of Jasminum flower extract. In some embodiments, the composition may include about 7% witch hazel extract. In some embodiments, the composition may include about 30% by weight of Pistacia lentiscus extract.

According to some embodiment, the composition comprises a carrier acceptable for topical administration.

In some embodiments, the composition is a pharmaceutical composition comprising one or more pharmaceutically acceptable excipients and/or carriers.

In some embodiments, the composition or the pharmaceutical composition is formulated for localized administration. In some embodiments, the localized administration is topical administration at the anus region, preferably on the affected region. In some embodiments, the formulation is in the form of a paste, a gel, a cream, an ointment, oily solution or suppository.

According to another aspect, the composition of the present invention is for use in treating an anorectal disorder. According to one embodiment, the anorectal disorder is hemorrhoids. In some embodiments, the hemorrhoids are internal or external. Thus, according to some embodiments, the composition comprising from about 1 to about 40% by weight of CBD oil, from about 5 to about 40% by weight of Jasminum flower extract, from about 5 to about 60% by weight of Pistacia lentiscus extract, from about 1 to about 35% by weight pomegranate peel powder and from about 1 to 25% by weight of witch hazel extract is for use in treating the anorectal disorder such as hemorrhoids. According to other embodiments, the composition comprising from about 1 to about 40% by weight of CBD oil, from about 5 to about 40% by weight of Jasminum flower extract, from about 5 to about 60% by weight of Pistacia lentiscus extract, from about 1 to about 35% by weight pomegranate peel powder and from about 1 to 35% by weight of Calendula flower extract is for use in treating the anorectal disorder such as hemorrhoids.

According to some embodiments, there is provided a method for treating anorectal disorder such as hemorrhoids in a subject in need thereof, the method comprising administering to the subject a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight of pomegranate peel powder, and from about 2.5 to about 15% by weight of witch hazel extract, to thereby treat the subject. According to other embodiments, there is provided a method for treating anorectal disorder such as hemorrhoids in a subject in need thereof, the method comprising administering to the subject a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight of pomegranate peel powder, and from about 5 to about 25% by weight of Calendula flower extract, to thereby treat the subject.

According to some embodiments, there is provided a method for treating anorectal disorder such as hemorrhoids in a subject in need thereof, the method comprising administering to the subject a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight of pomegranate peel powder, from about 2.5 to about 15% by weight of witch hazel extract, and optionally from about 2.5 to about 25% by weight of Calendula flower extract, from about 2.5 to about 25% by weight of Achillea millefolium extract, from about 2.5 to about 25% by weight of Ruscus aculeatus root extract, to thereby treat the subject. According to some embodiments, there is provided a method for treating anorectal disorder such as hemorrhoids in a subject in need thereof, the method comprising administering to the subject a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight of pomegranate peel powder, from about 2.5 to about 25% by weight of Calendula flower extract, and optionally from about 2.5 to about 15% by weight of witch hazel extract, from about 2.5 to about 25% by weight of Achillea millefolium extract, from about 2.5 to about 25% by weight of Ruscus aculeatus root extract, to thereby treat the subject.

Other objects, features and advantages of the present invention will become clear from the following description.

### DETAILED DESCRIPTION OF THE INVENTION

According to some embodiments, there are provided compositions and methods for treating or ameliorating an anorectal disorder such as hemorrhoids in a subject in need thereof. The compositions and methods disclosed herein are particularly efficient, useful and safe with diminished side effects, as demonstrated herein. Localized administration of the compositions result in an efficient and rapid improvement of the hemorrhoids condition, by providing a synergistic effect of the advantageous composition.

According to one aspect, the present invention provides a composition comprising from about 1 to about 40% by weight of CBD oil, from about 5 to about 40% by weight of Jasminum flower extract, from about 5 to about 60% by weight of Pistacia lentiscus extract, from about 1 to about 35% by weight pomegranate peel powder and from about 1 to about 35% by weight of witch hazel extract or Calendula flower extract. According to one embodiment, the composition comprises from about 1 to about 40% by weight of CBD oil, from about 5 to about 40% by weight of Jasminum flower extract, from about 5 to about 60% by weight of Pistacia lentiscus extract, from about 1 to about 35% by weight pomegranate peel powder and from about 1 to about 35% by weight of witch hazel extract. According to another embodiment, the composition comprises from about 1 to about 40% by weight of CBD oil, from about 5 to about 40% by weight of Jasminum flower extract, from about 5 to about 60% by weight of Pistacia lentiscus extract, from about 1 to about 35% by weight pomegranate peel powder and from about 1 to about 35% by weight of Calendula flower extract.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

The term “cannabinoid” as used herein generally refers to one of a class of diverse chemical compounds that act on a cannabinoid receptor in cells that repress neurotransmitter release in the brain. The term “cannabinoid” as used herein further refers a chemical compounds that acts on cannabinoid receptors or has a structure similar the stature of a compound acting on cannabinoid receptor in cells. Ligands for these receptor proteins include the endocannabinoids (produced naturally in the body by humans and animals), the phytocannabinoids (found in cannabis and some other plants), and synthetic cannabinoids (manufactured artificially).

The term “extract” as used herein refers a product prepared by extraction by physical means (e.g. by comminuting, pressing, heating, pulsed electric field assisted treatments, shear treatments and pressure wave treatments), by chemical means (e.g. by treatment with an acid, a base, a solvent) and/or by biochemical means (e.g. by treatment with hydrolytic enzymes, microorganisms). The term refers to a liquid substance obtained through extraction from a given substance, or to a concentrate or essence, which is free of, or substantially free of solvent. The term extract may be a single extract obtained from a particular extraction step or series of extraction steps. Extract also may be a combination of extracts obtained from separate extraction steps or separate feedstocks. Such combined extracts are thus also encompassed by the term “extract”. Any method of extraction and any solvent is encompassed. The extract may be obtained from any part of the plant e.g. from leaves, flowers, stems, roots, fruits and seeds. The extract may be aqueous or oily. Commonly used solvents are water, ethanol, ethyl acetate, CO2, methanol, acetone, and acetic acid. According to one embodiment, the solvent is ethanol. According to another embodiment, the extraction is by CO2. In particular, the term “extract” refers to a liquid or semi-solid or resinous substance obtained through extraction from plants defined in the present application, i.e. extracts obtained from cannabis plant e.g. CBD-rich strain, Jasminum flowers, Pistacia lentiscus, witch hazel, Calendula flowers, Achillea millefolium, and Ruscus aculeatus. In some embodiments, the term refers to a mixture of liquid or semi-solid, resinous substances obtained through extraction from two or more different plans. In some embodiments, the term refers also to a compound purified from the extract. According to some embodiments, the term “extract” has the meaning of a mixture or combination of two or more extracts. The extract also may be formulated into a pharmaceutical composition or food product, as described further below.

The term “cannabis extract” as used herein refers to one or more plant extracts from the cannabis plant. A cannabis extract contains, in addition to one or more cannabinoids, one or more non-cannabinoid components, which are co-extracted with the cannabinoids from the plant material. Their respective ranges in weight will vary according to the starting plant material and the extraction methodology used. Cannabinoid-containing plant extracts may be obtained by various means of extraction of cannabis plant material. Such means include but are not limited to: supercritical or subcritical extraction with CO2, extraction with hot or cold gas and extraction with solvents. In some embodiments, the term refers to a mixture of liquid or semi-solid, resinous substances obtained through extraction from two or more different cannabis species. In some embodiments, the term refers also to a compound purified from the extract. The term “cannabis plant” as used herein, refers to plants of the genus Cannabis, including but not limited to Cannabis sativa, Cannabis indica, and Cannabis ruderalis. According to one embodiment, the cannabis plant is a CBD-rich strain of cannabis plant.

The term “natural cannabinoid” as used herein generally refers to a cannabinoid, which can be found in, isolated from and/or extracted from a natural resource, such as plants. “Synthetic cannabinoids” are a class of chemicals that are different from the cannabinoids found e.g. in cannabis but which also bind to cannabinoid receptors.

In certain embodiments, the cannabinoid is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV) and cannabigerol monomethyl ether (CBGM), salts thereof, derivatives thereof and mixtures of cannabinoids. Each possibility represents a separate embodiment of the invention.

In some embodiments, the pharmacologically active cannabinoid may be selected from the group consisting of cannabidiol (CBD), tetrahydrocannabinol, Δ9-tetrahydrocannabinol (THC), Δ8-tetrahydrocannabinol, standardized marijuana extracts, Δ8-tetrahydrocannabinol-DMH, Δ9-tetrahydrocannabinol propyl analogue (THCV), 11-hydroxy-tetrahydrocannabinol, 11-nor-9-carboxy-tetrahydrocannabinol, 5′-azido-Δ8-tetrahydrocannabinol, AMG-1 (CAS Number 205746-46-9), AMG-3 (CAS Number 205746-46-9), AM-411 (CAS Number 212835-02-4), (−)-11-hydroxy-7′ sothiocyanato-Δ8-THC (AM-708), (−)-11-hydroxy-7′-azido-Δ8-THC (AM-836), AM-855 (CAS Number 249888-50-4), AM-919 (CAS Number 164228-46-0), AM926, AM-938 (CAS Number 303113-08-8), cannabidiol (CBD), cannabidiol propyl analogue (CBDV), cannabinol (CBN), cannabichromene, cannabichromene propyl analogue, cannabigerol, CP 47,497 (CAS Number (1S,3R): 114753-51-4), CP 55,940 (CAS Number 83002-04-4), CP 55,244 (CAS Number 79678-32-3), CT-3 (ajulemic acid), dimethylheptyl HHC, HU-210 (1,1-Dimethylheptyl-11-hydroxy-tetrahydrocannabinol), HU-211 (CAS Number 112924-45-5), HU-308 (CAS Number 1220887-84-2), WIN 55212-2 (CAS Number 131543-22-1), desacetyl-L-nantradol, dexanabinol, JWH-051 (Formula C25H3802), levonantradol, L-759633 (Formula C26H4002), nabilone, 0-1184, and mixtures thereof. Each possibility is a separate embodiment.

The terms “cannabidiol” and “CBD” are interchangeably used herein and refer to a non-psychotropic cannabinoid having structure as described in Formula I below, salt or derivatives thereof, such as Δ4-cannabidiol, Δ5-cannabidiol, Δ6-cannabidiol, Δ1,7-cannabidiol, Δ1-cannabidiol Δ2-cannabidiol Δ3-cannabidiol.

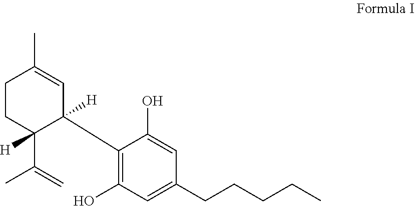 [SMILES: C=C(C)[C@@H]1CCC(C)=C[C@H]1c1c(O)cc(CCCCC)cc1O]

As used herein the term “CBD oil” is directed to a lipophilic plant extract which comprises CBD and optionally one or more additional cannabinoids, such as, for example, but not limited to: THC, CBN, and the like. The term CBD Oil further encompasses oil formulations including pure cannabinoids (for example, synthetic forms) including CBD and optionally one or more additional cannabinoids. According to some embodiments, the term “CBD oil” encompasses also crude extract of cannabis, in particular from a CBD rich strain, a fraction of the cannabis extract comprising high concentration of CBD, or purified CBD. According to some embodiments, the CBD oil comprises less than 5 wt %, less than 3 wt %, less than 2 wt %, 1 wt %, less than 0.5 wt %, less than 0.2 wt % or less than 0.1 wt % of THC. According to some embodiments, the CBD oil comprises from 1 to 3.5, from 1.5 to 3.5 wt % or from 2 to 3 wt % of THC. According to one embodiment, the CBD oil comprises from 55 to 65 wt % of CBD and or from 2 to 3 wt % of THC. According to some such embodiments, the CBD:THC weight ratio in the CBD oil is at least 10:1, at least 15:1 or at least 20:1. According to some embodiment, the CBD:THC weight ratio is from 20:1 to 1:1 or from 20:1 to 1:1.

The term “pharmaceutical composition” as used herein has its conventional meaning and refers to a composition, which is pharmaceutically acceptable. The term “pharmaceutically acceptable” as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. The term “excipient” as used herein has its conventional meaning and refers to a pharmaceutically acceptable ingredient, which is commonly used in the pharmaceutical technology for preparing a suitable topical dosage formulation.

The term “pharmaceutically acceptable carrier” or “pharmaceutically acceptable excipient” includes any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The term “carrier” refers to a diluent, adjuvant, excipient, or vehicle with which the therapeutic is administered. Such carriers can be sterile liquids, such as water or oils, including those of petroleum, animal, vegetable or synthetic origin, such as peanut oil, soybean oil, mineral oil, sesame oil and the like, polyethylene glycols, glycerine, propylene glycol or other synthetic solvents. According to some embodiments, the carrier is a pharmaceutically acceptable carrier.

The terms “topically acceptable carrier” and “dermatologically acceptable carrier” are used herein interchangeably and refer to any physiologically acceptable carrier suitable and safe for topical administration to mammals and in particular to human. According to some embodiment, the topically acceptable carrier is generally recognized as safe (GRAS). The term is intended to mean those carriers, which can safely be used on the skin or anorectal area in the topical treatment methods of this invention, i.e., carriers which do not provoke a severe reaction and which are not toxic when contacted with the skin. According to some embodiment, the topically acceptable carrier is a pharmaceutically acceptable carrier.

The term “topical administration” or “topical application” refers to directly layering or spreading upon epidermal tissue, especially outer skin or membrane, including the tissue of rectum and anus. The term comprises also intra-rectal and intra-anal administration of solids and semi-solid compositions, such as suppositories, creams and ointments.

The term “dosage form” denotes any form of the formulation that contains an amount of a cannabinoid or of a mixture of cannabinoids sufficient to achieve at least a partial therapeutic effect when appropriately administered. In some embodiments, the dosage form is for localized, topical formulation, including, but not limited to: oily liquid, solution, ointment, paste, gel, lotion, cream, suppository, or combinations thereof.

The terms “treat,” “treating,” or “treatment” as used herein, include reducing, alleviating, ablating, ameliorating, relieving, or lessening a symptom associated with either a chronic or an acute therapeutic scenario treatable with the composition of the invention. In some embodiments, the term includes, but is not limited to, alleviation or amelioration of one or more symptoms or parameters associated with hemorrhoids, such as improvement in parameters as assessed by various rating scales, tests, appearance and/or indices. In some embodiments, the term relates to: reducing, ameliorating and/or eliminating the symptoms of anorectal disorder e.g. reducing the hemorrhoids conditions and/or the symptoms associated therewith, such as, swelling, irritation, itching, bleeding, and the like.

The term “hemorrhoids” is directed to include pathological condition in the anus-rectal canal (anorectal disease) resulting from the swelling or inflammation or any other abnormal function or appearance of the hemorrhoids. In some embodiments, the hemorrhoids are internal. In other embodiments, the hemorrhoids are external.

The term “about” as used herein refers to any value, which lies within a range of ±10% of original value.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In some embodiments, the cannabinoid is a natural cannabinoid. In some embodiments, the cannabinoid is a natural cannabinoid found in a Cannabis plant. In some embodiments, the cannabinoid is a synthetic cannabinoid. In some embodiments, the cannabinoid is a mixture of natural cannabinoids. In some embodiments, the cannabinoid is a mixture of synthetic cannabinoids. In some embodiments, the cannabinoid is a mixture of natural and synthetic cannabinoids.

In some embodiments, the CBD oil may include from about 2% to about 90% CBD by weight. In some embodiments, the CBD oil may include from about 2% to about 75% CBD by weight. In some embodiments, the CBD oil may include from about 2% to about 60% CBD, by weight. In some embodiments, the CBD oil may include from about 2% to about 50% CBD. In some embodiments, the CBD oil may include from about 2% to about 40% CBD by weight. In some embodiments, the CBD oil may include from about 2% to about 30% CBD by weight. In some embodiments, the CBD oil may include from about 2% to about 20% CBD by weight. In some embodiments, the CBD oil may include from about 2% to about 15% CBD by weight. In some embodiments, the CBD oil may include from about 5% to about 75% CBD by weight. In some embodiments, the CBD oil may include from about 5% to about 50% CBD by weight. In some embodiments, the CBD oil may include from about 5% to about 30% CBD by weight. In some embodiments, the CBD oil may include from about 5% to about 30% CBD by weight. In some embodiments, the CBD oil may include from about 5% to about 20% CBD by weight. In some embodiments, the CBD oil may include from about 7.5% to about 25% CBD by weight. In some embodiments, the CBD oil may include from about 7.5% to about 20% CBD by weight. In some embodiments, the CBD oil may include from about 7.5% to about 15% CBD by weight. In some embodiments, the CBD oil may include from about 10% to about 20% CBD by weight. In some embodiments, the CBD oil may include from about 20% to about 40% CBD by weight. In some embodiments, the CBD oil may include from about 30% to about 60% CBD by weight. In some embodiments, the CBD oil may include from about 50% to about 90% CBD by weight. In some embodiments, the CBD oil may include from about 60% to about 80% CBD by weight. According to some embodiments, CBD oil comprises from 20 to 40 wt %, from 40 to 80 wt %, from 55 to 80 wt %, from 50 to 70 wt % or from 60 to 75 wt % CBD. According to some embodiments, the CBD oil comprises from 55 to 80 wt % of CBD. According to some embodiments, the CBD oil comprises from 55 to 65 wt % of CBD. According to some embodiments, the CBD oil comprises from 40 to 80 wt % of CBD. According to some embodiments, the CBD oil comprises less than 5 wt %, less than 3 wt %, less than 2 wt %, 1 wt %, less than 0.5 wt %, less than 0.2 wt % or less than 0.1 wt % of THC. According to some embodiments, the CBD oil comprises from 1 to 3.5, from 1.5 to 3.5 wt % or from 2 to 3 wt % of THC. According to one embodiment, the CBD oil comprises from 55 to 65 wt % of CBD and or from 2 to 3 wt % of THC. According to some such embodiments, the CBD:THC weight ratio in the CBD oil is at least 10:1, at least 15:1 or at least 20:1. According to some embodiment, the CBD:THC weight ratio is from 20:1 to 1:1 or from 20:1 to 1:1.

In some embodiments, the natural cannabinoid such as CBD is derived or isolated from an extract of a Cannabis plant. In some embodiments of the composition, the composition further comprises natural terpene(s) derived or isolated from an extract of a Cannabis plant.

In some embodiments, the pharmaceutical composition or the CBD oil may further include one or more cannabinoids in addition to CBD. In some embodiments, the one or more cannabinoids are selected from the group consisting of Cannabidiolic acid (CBDA), Tetrahydrocannabinol (THC), Tetrahydrocannabinolic acid (THCA), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM), salts thereof, and derivatives thereof.

In some embodiments, the composition or the CBD oil comprises about 30% to about 80% by weight of a cannabinoid or a mixture of cannabinoids. In certain embodiments, the composition or the CBD oil comprises about 40% to about 70% by weight of a cannabinoid or a mixture of cannabinoids. In certain embodiments, the cannabinoid is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives thereof and mixtures of cannabinoids.

In some embodiments, the amounts of the active ingredients may be determined by the skilled artisan accordingly to meet the desired concentration in the final product. Thus, for example, the concentration of CBD (e.g. in the oil used as a starting material) may be determined such that the final composition may contain about 0.5 to about 32%, about 1 to about 30%, about 2 to about 25%, about 3 to about 20%, about 4 to about 17%, about 0.5 to about 8%, about 1 to about 5%, about 1 to about 3%, 3 to about 9%, about 4 to about 8% e.g. 1.8% or 5% CBD by weight. According to some embodiments, the composition comprises from about 2 to about 9 wt %, from about 2 to about 8 wt %, from about 3 to about 8 wt %, from about 3 to about 7 wt % or from about 4 to about 6 wt % of CBD. According to some embodiment, the composition comprises from 2 to 25 wt %, from 3 to 22.5 wt %, from 4 to 20 wt %, from 5 to 15 wt %, or from 2 to 16 wt % CBD.

According to some embodiments, the composition of the invention may include about 1 to about 35% CBD oil by weight. According to some embodiments, the composition of the invention may include about 2 to about 25% CBD oil by weight. According to some embodiments, the composition of the invention may include about 5 to about 30% CBD oil by weight. According to some embodiments, the composition of the invention may include about 5 to about 25% CBD oil by weight. According to other embodiments, the composition of the invention may include about 5 to about 22% CBD oil by weight. According to some embodiments, the composition of the invention may include about 5 to about 20% CBD oil by weight. According to some embodiments, the composition of the invention may include about 7.5 to about 20% CBD oil by weight. According to some embodiments, the composition of the invention may include about 8 to about 15% CBD oil by weight. According to some embodiments, the composition of the invention may include about 10 to 14% or about 9 to about 13% CBD oil by weight. In some exemplary embodiments, the composition of the invention may include about 12% CBD oil by weight. According to some embodiments, the composition comprises from 3 to 9 wt %, from 8 to about 20%, from 8 to about 15 wt % or 8%, 9%, 10%, 11%, 12%, 13%, 14%, 15%, 16%, 17%, 18%, 19%, 20%, 21%, 22%, 23% or 24% by weight of CBD oil. According to some embodiments, CBD oil comprises from 20 to 40 wt %, from 40 to 80 wt %, from 30 to 60 wt %, from 55 to 80 wt %, from 50 to 70 wt % or from 60 to 75 wt % CBD. According to some embodiments, CBD oil comprises from 55 to 65 wt % CBD.

In some embodiments, the composition comprises at least about 20 mg of a cannabinoid or a mixture of cannabinoids, per 1 gr of composition. In some embodiments, the composition comprises at least about 50 mg of a cannabinoid or a mixture of cannabinoids, per 1 gr of composition. In some embodiments, the composition comprises about 20 mg to about 800 mg of a cannabinoid or a mixture of cannabinoids, per 1 gr of composition. In some embodiments, the composition comprises about 50 mg to about 500 mg of a cannabinoid or a mixture of cannabinoids, per 1 gr of composition. In some embodiments, the composition comprises at least about 20 to about 500 mg of a cannabinoid or a mixture of cannabinoids, per 1 gr of composition. In some embodiments, the composition comprises at least about 10 to about 300 mg, from 30 to 250, or from 50 to 200 of a cannabinoid or a mixture of cannabinoids, per 1 gr of composition. In some embodiments, the composition comprises at least about 40 to about 120 mg of a cannabinoid or a mixture of cannabinoids, per 1 gr of composition. In some embodiments, the composition comprises at least about 60 to about 110 mg of a cannabinoid or a mixture of cannabinoids, per 1 gr of composition. In some embodiments, the composition comprises at least about 60 to about 80 mg of a cannabinoid or a mixture of cannabinoids, per 1 gr of composition. In some embodiments, the composition comprises about 20 mg, about 50 mg, about 100 mg, about 200 mg, about 300 mg, about 400 mg, about 500 mg or about 600 mg of a cannabinoid or a mixture of cannabinoids, per 1 gr of composition.

In some embodiments, one or more cannabinoids may be added to the composition in purified form. For example, CBD-containing oil may be manufactured synthetically, by addition of CBD (and optionally other cannabinoids and/or cannabis-derived compounds such as terpenes) to oil, which may subsequently be used in manufacturing the composition.

According to embodiments of the invention, the composition may further include pomegranate peel powder. Typically, pomegranate peel powder may be obtained/extracted from the peels. Pomegranate Peel Powder has anti-oxidant effect, owing to the tannins and Bioflavonoids present. Tanis can further act as a vasoconstrictor. Bioflavonoids can protect blood vessels, maintain their flexibility and prevent blood leaking therefrom. According to some embodiments, the composition may include about 2.5 to about 25%, by weight of pomegranate peel powder. According to some embodiments, the composition may include about 5 to about 20% by weight of pomegranate peel powder. According to some embodiments, the composition may include about 7.5 to about 15% by weight of pomegranate peel powder. According to some embodiments, the composition may include about 8 to about 12% by weight of pomegranate peel powder. According to some embodiments, the composition may include about 10% by weight pomegranate peel powder.

According to some embodiments, the composition may further include Pistacia lentiscus extract. Typically, Pistacia lentiscus extract may be obtained/extracted from the resin/leaves/fruits. Pistacia lentiscus has anti-inflammatory properties and selective antibacterial activity. It is also an antioxidant. According to some embodiments, the composition may include from about 5 to about 60% by weight of Pistacia lentiscus. According to some embodiments, the composition may include about 10 to about 50% by weight of Pistacia lentiscus. According to some embodiments, the composition may include about from 15 to about 40% by weight of Pistacia lentiscus. According to some embodiments, the composition may include about from 20 to about 40% by weight of Pistacia lentiscus. According to other embodiments, the composition may include about from 25 to about 35% by weight of Pistacia lentiscus. According to some embodiments, the composition may include about 30% by weight Pistacia lentiscus extract.

According to some embodiments, the composition may further include Jasminum flower extract. Typically, Jasminum flower extract may be obtained/extracted from the flowers. According to some embodiments, the composition may include about 5% to about 40% by weight of Jasminum flower extract. According to some embodiments, the composition may include about 10 to 30 Jasminum flower extract. According to some embodiments, the composition may include about 7 to about 35% by weight of Jasminum flower extract According to some embodiments, the composition may include about 15 to about 20% by weight of Jasminum flower extract. According to some embodiments, the composition may include about 10 to about 25% by weight of Jasminum flower extract. According to some embodiments, the composition may include from about 12 to about 22% or from about 15 to about 20% by weight of Jasminum flower extract. According to some embodiments, the composition may include about 17% by weight Jasminum flower extract.

According to some embodiments, the composition may further include witch hazel extract. Typically, witch hazel extract may be obtained/extracted from the leaves and barks. Witch hazel includes tannins, which possess stringent and anti-hemorrhoid activities. According to some embodiments, the composition may include about 1 to about 25 wt %, from about 2.5 to about 25% by weight of witch hazel. According to some embodiments, the composition may include from about 5 to about 20% by weight of witch hazel. According to some embodiments, the composition may include about 7.5 to about 15% by weight of witch hazel. According to some embodiments, the composition may include about 7% or 15% by weight witch hazel extract. According to some embodiments, the composition comprises from about 3 to about 15 wt % of witch hazel extract. According to some embodiments, the composition comprises from about 4 to about 12 wt % of witch hazel extract. According to some embodiments, the composition comprises from about 5 to about 10 wt % of witch hazel extract.

According to some embodiments, the composition may further optionally include Calendula flower extract. Typically, Calendula flower extract may be obtained/extracted from the plant flower. According to some embodiments, the composition may include from about 1 to about 25 wt % or from about 2.5 to about 25% by weight of Calendula flower extract. According to some embodiments, the composition may include from about 5 to about 20 wt % of Calendula flower extract. According to some embodiments, the composition may include from about 7.5 to about 15% by weight of Calendula flower extract. According to some embodiments, the composition may include from about 8 to about 12% by weight of Calendula flower extract. According to some embodiments, the composition may include about 10% by weight of Calendula flower extract. According to some embodiments, the composition comprises from about 3 to about 15 wt % of Calendula flower extract. According to some embodiments, the composition comprises from about 4 to about 12 wt % Calendula flower extract. According to some embodiments, the composition comprises from about 5 to about 12 wt % of Calendula flower extract.

According to some embodiments, the composition may further optionally include Achillea millefolium extract. Typically, Achillea millefolium extract may be obtained/extracted from the leaves and flowers. According to some embodiments, the composition may include from about 1 to about 25 wt %, or from about 2.5 to about 25% by weight of Achillea millefolium extract. According to some embodiments, the composition may include about 4-20% by weight of Achillea millefolium extract. According to some embodiments, the composition may include from 4 to about 20% by weight of Achillea millefolium extract. According to some embodiments, the composition may include from about 6 to about 15% by weight of Achillea millefolium extract. According to some embodiments, the composition may include about 7% by weight of Achillea millefolium extract. According to some embodiments, the composition comprises from about 3 to about 15 wt % of Achillea millefolium extract. According to some embodiments, the composition comprises from about 4 to about 12 wt % Achillea millefolium extract. According to some embodiments, the composition comprises from about 5 to about 10 wt % of Achillea millefolium extract.

According to some embodiments, the composition may further optionally include Ruscus aculeatus root extract. Typically, Ruscus aculeatus root extract may be obtained/extracted from the root. According to some embodiments, the composition may include from about 1 to about 25 wt %, from about 2.5 to about 25% by weight of Ruscus aculeatus root extract. According to some embodiments, the composition may include from 4 to about 20% by weight of Ruscus aculeatus root extract. According to some embodiments, the composition may include from about 6 to about 15% by weight of Ruscus aculeatus root extract. According to some embodiments, the composition may include about 7% by weight of Ruscus aculeatus root extract. According to some embodiments, the composition comprises from about 3 to about 15 wt % of Ruscus aculeatus root extract. According to some embodiments, the composition comprises from about 4 to about 12 wt % Ruscus aculeatus root extract. According to some embodiments, the composition comprises from about 5 to about 10 wt % of Ruscus aculeatus root extract.

According to some embodiments, there composition may further optionally include 10 to about 95% cocoa butter. According to some embodiments, the composition comprises from 15 to 90 wt %, from 20 to 85 wt %, from 25 to 80 wt %, from 30 to 75 wt %, from 35 to 70 wt %, from 40 to 65 wt %, from 45 to 60 wt %, from 50 to 55 wt %, from 55 to 90 wt %, from 60 to 85 wt %, from 65 to 80 wt % of cocoa butter.

According to some embodiments, there is thus provided a composition comprising from about 5 to about 30% by weight of CBD oil, from about 15 to about 30% by weight of Jasminum flower extract, from about 15 to about 50% by weight of Pistacia lentiscus extract, from about 5 to about 45% by weight pomegranate peel powder and from about 2.5 to about 45% by weight of Calendula flower extract. According to some embodiments, the composition comprises from about 10 to about 20% by weight of CBD oil, from about 15 to about 30% by weight of Jasminum flower extract, from about 15 to about 30% by weight of Pistacia lentiscus extract, from about 15 to about 30% by weight pomegranate peel powder and from about 15 to about 30% by weight of Calendula flower extract.

According to some embodiments, there is thus provided a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight pomegranate peel powder, from about 2.5 to about 15% by weight of witch hazel extract; and optionally from about 2.5 to about 15% by weight of one or more of each of: Calendula flower extract, Achillea millefolium extract and/or Ruscus aculeatus root extract. According to some embodiments, there is thus provided a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight pomegranate peel powder, from about 2.5 to about 15% by weight of Calendula flower extract; and optionally from about 2.5 to about 15% by weight of one or more of each of: witch hazel extract, Achillea millefolium extract and/or Ruscus aculeatus root extract.

According to one embodiments, there is provided a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight pomegranate peel powder, from about 2.5 to about 15% by weight of witch hazel extract; and from about 2.5 to about 15% by weight of Calendula flower extract. According to some embodiments, there is provided a composition comprising from about 5 to about 30% by weight of CBD oil, from about 10 to about 30% by weight of Jasminum flower extract, from about 15 to about 50% by weight of Pistacia lentiscus extract, from about 5 to about 25% by weight pomegranate peel powder, from about 2.5 to about 15% by weight of witch hazel extract, from about 2.5 to about 15% by weight of Calendula flower extract, and from about 2.5 to about 15% by weight of Achillea millefolium extract or Ruscus aculeatus root extract. According to some embodiments, there is provided a composition comprising from about 5 to about 30% by weight of CBD oil, from about 10 to about 30% by weight of Jasminum flower extract, from about 15 to about 50% by weight of Pistacia lentiscus extract, from about 5 to about 25% by weight pomegranate peel powder, from about 2.5 to about 15% by weight of witch hazel extract, from about 2.5 to about 15% by weight of Achillea millefolium extract, and from about 2.5 to about 15% by weight of Calendula flower extract or Ruscus aculeatus root extract. According to some embodiments, the composition comprises from 15 to 30 wt % of Calendula flower extract. According to some embodiments, there is provided a composition comprising from about 5 to about 30% by weight of CBD oil, from about 10 to about 30% by weight of Jasminum flower extract, from about 15 to about 50% by weight of Pistacia lentiscus extract, from about 5 to about 25% by weight pomegranate peel powder, from about 2.5 to about 15% by weight of Calendula flower extract, from about 2.5 to about 15% by weight of Achillea millefolium extract, and from about 2.5 to about 15% by weight of witch hazel extract or Ruscus aculeatus root extract.

According to some embodiments, there is provided a composition comprising from about 5 to about 30% by weight of CBD oil, from about 10 to about 30% by weight of Jasminum flower extract, from about 15 to about 50% by weight of Pistacia lentiscus extract, from about 5 to about 25% by weight pomegranate peel powder, from about 2.5 to about 15% by weight of witch hazel extract, from about 2.5 to about 15% by weight of Calendula flower extract, from about 2.5 to about 15% by weight of Achillea millefolium extract and from about 2.5 to about 15% Ruscus aculeatus root extract.

According to one embodiment, the present invention provides a composition comprising or consisting of from about 5 to about 30% by weight of CBD oil, from about 15 to about 30% by weight of Jasminum Flower extract, from about 15 to about 50% by weight of Pistacia lentiscus extract, from about 5 to about 25% by weight of pomegranate peel powder, from about 2.5 to about 15% by weight of witch hazel extract, from about 2.5 to about 25% by weight of Calendula flower extract, from about 2.5 to about 25% by weight of Achillea millefolium extract, from about 2.5 to about 25% by weight of Ruscus aculeatus root extract.

The term “comprises” encompasses also the term “consisting of” and may be replaced by it in any embodiment of the present application. Thus, in one embodiment, the composition of the present invention consists of According to some embodiments, there is thus provided a composition consisting of from about 5 to about 30% by weight of CBD oil, from about 15 to about 30% by weight of Jasminum flower extract, from about 15 to about 50% by weight of Pistacia lentiscus extract, from about 5 to about 25% by weight pomegranate peel powder, from about 2.5 to about 15% by weight of witch hazel extract; and optionally from about 2.5 to about 15% by weight of one or more of each of: Calendula flower extract, Achillea millefolium extract and/or Ruscus aculeatus root extract and optionally a pharmaceutically acceptable excipient and/or carrier. According to some embodiments, there is thus provided a composition consisting of from about 5 to about 30% by weight of CBD oil, from about 15 to about 30% by weight of Jasminum flower extract, from about 15 to about 50% by weight of Pistacia lentiscus extract, from about 5 to about 25% by weight pomegranate peel powder, from about 2.5 to about 15% by weight of Calendula flower extract; and optionally from about 2.5 to about 15% by weight of one or more of each of: witch hazel extract, Achillea millefolium extract and/or Ruscus aculeatus root extract and optionally a pharmaceutically acceptable excipient and/or carrier. According to another embodiment, the composition of the present invention consists of from about 5 to about 30% by weight of CBD oil, from about 15 to about 30% by weight of Jasminum flower extract, from about 15 to about 50% by weight of Pistacia lentiscus extract, from about 5 to about 25% by weight pomegranate peel powder, from about 2.5 to about 15% by weight of witch hazel extract, from about 2.5 to about 15% by weight of Calendula flower extract, and from about 2.5 to about 15% by weight of Achillea millefolium extract and 2.5 to about 15% Ruscus aculeatus root extract and optionally a pharmaceutically acceptable excipient and/or carrier.

According to some embodiments, the composition is anti-hemorrhoid composition.

According to some embodiments, there is provided a topical anti-hemorrhoidal composition comprising therapeutically effective amounts of one or more of:

a) Cannabis, in the form of CBD oil;
b) pomegranate peel powder;
c) Jasminum flower extract;
d) Pistacia lentiscus extract;
e) Calendula flower extract;
f) Optionally, witch hazel extract
g) Optionally, Achillea millefolium extract; and
h) Optionally, Ruscus aculeatus extract.

Each possibility is a separate embodiment.

According to some embodiments, there is provided a topical anti-hemorrhoidal composition comprising therapeutically effective amounts of one or more of: Cannabis, in the form of CBD oil; pomegranate peel powder; Jasminum flower extract; Pistacia lentiscus extract; witch hazel extract; Optionally Calendula flower extract; Optionally, Achillea millefolium extract; and Optionally, Ruscus aculeatus extract. Each possibility is a separate embodiment.

In some embodiments there is provided a topical composition comprising:

a) 1 to about 30% by weight of CBD oil;
b) 5 to about 25% by weight of pomegranate peel powder;
c) 15 to about 30% by weight of Jasminum flower extract;
d) 15 to about 50% by weight of Pistacia lentiscus extract;
e) 2.5 to about 15% by weight witch hazel extract
f) Optionally, 2.5 to about 20% by weight of Calendula flower extract
g) Optionally 2.5 to about 15% by weight of Achillea millefolium extract; and
h) Optionally 2.5 to about 15% by weight of Ruscus aculeatus extract;

Each possibility is a separate embodiment.

In some embodiments there is provided a topical composition comprising: 1 to about 30% by weight of CBD oil; 5 to about 25% by weight of pomegranate peel powder; 15 to about 30% by weight of Jasminum flower extract; 15 to about 50% by weight of Pistacia lentiscus extract; 2.5 to about 20% by weight Calendula flower extract; optionally 2.5 to about 15% by weight of witch hazel extract; optionally 2.5 to about 15% by weight of Achillea millefolium extract; and optionally 2.5 to about 15% by weight of Ruscus aculeatus extract; Each possibility is a separate embodiment.

In some embodiments there is provided a topical composition comprising:

a) About 12% by weight of CBD oil;
b) About 10% by weight of pomegranate peel powder;
c) About 17% by weight of Jasminum flower extract;
d) About 30% by weight of Pistacia lentiscus extract;
e) About 7% by weight witch hazel extract
f) Optionally, about 10% by weight of Calendula flower extract
g) Optionally about 7% by weight of Achillea millefolium extract; and
h) Optionally, about 7% by weight of Ruscus aculeatus extract;

Each possibility is a separate embodiment.

According to some embodiments, the CBD oil comprises from 40 to 80 wt % of CBD. According to some embodiments, the CBD oil comprises from 30 to 60 wt % of CBD. According to some embodiments, the CBD oil comprises from 55 to 80 wt % of CBD. According to some embodiments, the CBD oil comprises from 55 to 65 wt % of CBD. According to some embodiments, the composition, such as a pharmaceutical composition, comprises from 1 to 10 or from 2 to 8 wt % CBD. According to some embodiment, the composition comprises from 2 to 25, from 3 to 22.5, from 4 to 20, from 5 to 15, or from 2 to 16 wt % CBD. According to some embodiments, the composition, such as a pharmaceutical composition, comprises from 4 to 15 or from 5 to 12 wt % CBD.

The composition according to any one of the above embodiments, further comprising a topically acceptable carrier. According to some embodiment, the topically acceptable carrier is any carrier suitable for topical administration to a human being. According to some embodiments, the topically acceptable carrier is a pharmaceutically acceptable carrier.

According to some embodiments, the composition is a pharmaceutical composition further comprising a pharmaceutically acceptable excipient and/or carrier.

According to some embodiments, the composition, e.g. a pharmaceutical composition is formulated for a local administration. According to one embodiment, the local administration is a topical administration. According to one embodiment, the composition, e.g. the pharmaceutical composition, is formulated as a paste, a gel, a cream, an ointment, or oily solution. According to other embodiments, the composition is formulated as a suppository. According to other embodiments, the composition is formulated as an ointment. According to other embodiments, the composition is formulated as a cream. According to some embodiments, the composition comprises from 20 to 120 mg of CBD. According to other embodiments, the composition comprises from 40 to 120 mg of CBD per 1 gram of a composition.

According to another aspect, the composition of the present invention is for use in treating an anorectal disorder. The term “anorectal disorder” as used herein includes any disorder associated with an anal rectal disease, including, but not limited to, acute or chronic anal fissures, internally or externally thrombosed hemorrhoids, hemorrhoidal diseases, disorders associated with endoscopic hemorrhoidal ligation, levator spasms, constipation, and pain associated with any anorectal disorder. According to one embodiment, the anorectal disorder is hemorrhoids. According to another embodiment, the anorectal disorder is anal fistula. According to another embodiment, the anorectal disorder is anal fissure. Thus, according to some embodiments, the composition according to any one of the above aspects and embodiments are for use in treating an anorectal disorder such as hemorrhoids. Thus according to one embodiment, the present invention provides a composition comprising from about 1 to about 40% by weight of CBD oil, from about 5 to about 40% by weight of Jasminum flower extract, from about 5 to about 60% by weight of Pistacia lentiscus extract, from about 1 to about 35% by weight pomegranate peel powder and from about 1 to 25% by weight of witch hazel extract, for use in treating an anorectal disorder. According to one embodiment, the anorectal disorder is hemorrhoids. According to another embodiment, the present invention provides a composition comprising from about 1 to about 40% by weight of CBD oil, from about 5 to about 40% by weight of Jasminum flower extract, from about 5 to about 60% by weight of Pistacia lentiscus extract, from about 1 to about 35% by weight pomegranate peel powder and from about 1 to 35% by weight of Calendula extract, for use in treating an anorectal disorder. According to one embodiment, the anorectal disorder is hemorrhoids.

According to another embodiment, the present invention provides a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight of pomegranate peel powder, and from about 2.5 to about 15% by weight of witch hazel extract, for use in treating an anorectal disorder such as hemorrhoids. According to one embodiment, the composition is a pharmaceutical composition. According to a further embodiment, the present invention provides a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight of pomegranate peel powder, and from about 2.5 to about 25% by weight of Calendula flower extract, for use in treating an anorectal disorder such as hemorrhoids. According to one embodiment, the composition is a pharmaceutical composition.

According to some embodiments, the composition for use comprises from 5 to 25 wt % or from 8 to 22 wt % or from 8 to 20 wt % CBD oil. According to some embodiments, the CBD oil comprises from 5 to 50 wt % of CBD. According to some embodiments, the CBD oil comprises from 40 to 80 wt % of CBD. According to some embodiments, the CBD oil comprises from 30 to 60 wt % of CBD. According to some embodiments, the CBD oil comprises from 55 to 80 wt % of CBD. According to some embodiments, the CBD oil comprises from 55 to 70 wt % of CBD. According to some embodiments, the CBD oil comprises from 55 to 65 wt % of CBD. According to some embodiments, the composition, such as a pharmaceutical composition, comprises from 1 to 10 or from 2 to 8 wt % CBD. According to some embodiment, the composition comprises from 2 to 25, from 3 to 22.5, from 4 to 20, from 5 to 15, or from 2 to 16 wt % CBD. According to some embodiment, the composition comprises from 5 to 10 wt % CBD.

According to some embodiments, the composition comprises from 5 to 40 wt %, from 7 to 35 wt %, from 10 to 30 wt %, or 12 to 25 wt % of Jasminum flower extract. According to some embodiments, the composition comprises from 5 to 60 wt %, from 10 to 55 wt %, from 15 to 50 wt %, from 15 to 45 wt %, from 20 to 40 wt %, or from 25 to 35 wt % of Pistacia lentiscus extract. According to some embodiments, the composition comprises from 5 to 20 wt %, or from 7 to 15 wt % of pomegranate peel extract. According to another embodiment, the composition comprises from about 3 to about 15% or from 5 to 12% by weight of Calendula flower extract.

According to one embodiment, the present invention provides a composition comprising from about 5 to about 20% by weight of CBD oil, from about 15 to about 35% by weight of Jasminum flower extract, from about 15 to about 45% by weight of Pistacia lentiscus extract, from about 15 to about 35% by weight of pomegranate extract, and from about 15 to about 35% by weight of Calendula flower extract, for use in treating an anorectal disorder such as hemorrhoids.

According to one embodiment, the present invention provides a composition comprising from about 5 to about 22% by weight of CBD oil, from about 10 to about 30% by weight of Jasminum flower extract, from about 20 to about 40% by weight of Pistacia lentiscus extract, from about 5 to about 15% by weight of pomegranate extract, and from about 5 to about 10% by weight of witch hazel extract, for use in treating an anorectal disorder such as hemorrhoids. According to another embodiment, the present invention provides a composition comprising from about 5 to about 22% by weight of CBD oil, from about 10 to about 30% by weight of Jasminum flower extract, from about 20 to about 40% by weight of Pistacia lentiscus extract, from about 5 to about 15% by weight of pomegranate extract, and from about 5 to about 15% by weight of Calendula flower extract, for use in treating an anorectal disorder such as hemorrhoids.

According to some embodiments, there is provided a composition for use in treatment of a anorectal disorder in a subject in need thereof, the composition including from about 5 to about 30% by weight of CBD oil, from about 15 to about 30% by weight of Jasminum flower extract, about 15 to about 50% by weight of Pistacia lentiscus extract, from about 5 to about 25% by weight pomegranate peel powder, about 2.5 to about 15% by weight of witch hazel extract; and optionally from about 2.5 to about 15% by weight of Calendula flower extract, optionally from about 2.5 to about 15% by weight of Achillea millefolium extract, and optionally about 2.5 to about 15% by weight of Ruscus aculeatus root extract. According to other embodiments, there is provided a composition for use in treatment of a anorectal disorder in a subject in need thereof, the composition including from about 5 to about 30% by weight of CBD oil, from about 15 to about 30% by weight of Jasminum flower extract, about 15 to about 50% by weight of Pistacia lentiscus extract, from about 5 to about 25% by weight pomegranate peel powder, about 2.5 to about 15% by weight of Calendula flower extract; and optionally from about 2.5 to about 15% by weight of witch hazel extract, optionally from about 2.5 to about 15% by weight of Achillea millefolium extract, and optionally about 2.5 to about 15% by weight of Ruscus aculeatus root extract. Each possibility is a separate embodiment. According to one embodiment, the anorectal disorder is hemorrhoids.

According to some embodiments, there is provided a composition for use in treatment of hemorrhoids in a subject in need thereof, the composition comprises or consists of rom about 5 to about 30% by weight of CBD oil, from about 15 to about 30% by weight of Jasminum Flower extract, from about 15 to about 50% by weight of Pistacia lentiscus extract, from about 5 to about 25% by weight pomegranate peel powder, from about 2.5 to about 15% by weight of witch hazel extract; and optionally from about 2.5 to about 15% by weight of Calendula flower extract, optionally from about 2.5 to about 15% by weight of Achillea millefolium extract, optionally from about 2.5 to about 15% by weight of Ruscus aculeatus root extract. Each possibility is a separate embodiment.

According to another embodiment, the present invention provides a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight of pomegranate peel powder, from about 2.5 to about 15% by weight of witch hazel extract, from about 2.5 to about 25% by weight of Calendula flower extract, from about 2.5 to about 25% by weight of Achillea millefolium extract, from about 2.5 to about 25% by weight of Ruscus aculeatus root extract, for use in treating anorectal disorder. According to one embodiment, the anorectal disorder is hemorrhoids. According to one embodiment, the composition is a pharmaceutical composition.

According to some embodiments, there is provided a composition for use in treatment of hemorrhoids in a subject in need thereof, the composition includes from about 5 to about 30% by weight of CBD oil, from about 15 to about 30% by weight of Jasminum Flower extract, from about 15 to about 50% by weight of Pistacia lentiscus extract, from about 5 to about 25% by weight pomegranate peel powder, from about 2.5 to about 15% by weight of witch hazel extract, from about 2.5 to about 15% by weight of Calendula flower extract, from about 2.5 to about 15% by weight of Achillea millefolium extract, and from about 2.5 to about 15% by weight of Ruscus aculeatus root extract.

According to some embodiments, there is provided a composition for use in treatment of hemorrhoids in a subject in need thereof, the composition comprises or consists of from about 5 to about 30% by weight of CBD oil, from about 15 to about 30% by weight of Jasminum Flower extract, from about 15 to about 50% by weight of Pistacia lentiscus extract, from about 5 to about 25% by weight pomegranate peel powder, from about 2.5 to about 15% by weight of witch hazel extract; from about 2.5 to about 15% by weight of Calendula flower extract, from about 2.5 to about 15% by weight of Achillea millefolium extract, and from about 2.5 to about 15% by weight of Ruscus aculeatus root extract.

According to some embodiments, there is provided a composition for use in treatment of hemorrhoids in a subject in need thereof, the composition includes about 12% (weight) of CBD oil, about 17% by weight of Jasminum Flower extract, about 30% by weight of Pistacia lentiscus extract, about 10% by weight pomegranate peel powder, about 7% by weight of witch hazel extract; and optionally about 10% by weight of Calendula flower extract, optionally about 7% by weight of Achillea millefolium extract, optionally about 7% by weight of Ruscus aculeatus root extract. Each possibility is a separate embodiment. According to some embodiments, there is provided a composition for use in treatment of hemorrhoids in a subject in need thereof, the composition includes about 12% (weight) of CBD oil, about 17% by weight of Jasminum Flower extract, about 30% by weight of Pistacia lentiscus extract, about 10% by weight pomegranate peel powder, about 10% by weight of Calendula flower; and optionally about 7% by weight of witch hazel extract, optionally about 7% by weight of Achillea millefolium extract, optionally about 7% by weight of Ruscus aculeatus root extract. Each possibility is a separate embodiment.

According to some embodiments, there is provided a composition for use in treatment of hemorrhoids in a subject in need thereof, the composition including about 12% by weight of CBD oil, about 17% by weight of Jasminum flower extract, about 30% by weight of Pistacia lentiscus extract, about 10% by weight pomegranate peel powder, about 7% by weight of witch hazel extract; and optionally about 10% by weight of Calendula flower extract, optionally about 7% by weight of Achillea millefolium extract, optionally about 7% by weight of Ruscus aculeatus root extract. According to some embodiments, there is provided a composition for use in treatment of hemorrhoids in a subject in need thereof, the composition including about 12% by weight of CBD oil, about 17% by weight of Jasminum flower extract, about 30% by weight of Pistacia lentiscus extract, about 10% by weight pomegranate peel powder, about 10% by weight of Calendula flower extract; and optionally about 7% by weight of witch hazel extract, optionally about 7% by weight of Achillea millefolium extract, optionally about 7% by weight of Ruscus aculeatus root extract.

According to some embodiments, there is provided a composition for use in treatment of hemorrhoids in a subject in need thereof, the composition comprises or consists of about 12% by weight of CBD oil, about 17% by weight of Jasminum Flower extract, about 30% by weight of Pistacia lentiscus extract, about 10% by weight pomegranate peel powder, about 7% by weight of witch hazel extract; and optionally about 10% by weight of Calendula flower extract, optionally about 7% by weight of Achillea millefolium extract, optionally about 7% by weight of Ruscus aculeatus root extract, and optionally a pharmaceutically acceptable carrier. According to some embodiments, there is provided a composition for use in treatment of hemorrhoids in a subject in need thereof, the composition comprises or consists of about 12% by weight of CBD oil, about 17% by weight of Jasminum Flower extract, about 30% by weight of Pistacia lentiscus extract, about 10% by weight pomegranate peel powder, optionally about 10% by weight of Calendula flower extract; and optionally about 7% by weight of witch hazel extract, about 7% by weight of Achillea millefolium extract, optionally about 7% by weight of Ruscus aculeatus root extract, and optionally a pharmaceutically acceptable carrier. Each possibility is a separate embodiment.

According to some embodiments, there is provided a composition for use in treatment of hemorrhoids in a subject in need thereof, the composition includes about 12% by weight of CBD oil, about 17% by weight of Jasminum flower extract, about 30% by weight of Pistacia lentiscus extract, about 10% by weight pomegranate peel powder, about 7% by weight of witch hazel extract; about 10% by weight of Calendula flower extract, about 7% by weight of Achillea millefolium extract, and about 7% by weight of Ruscus aculeatus root extract. Each possibility is a separate embodiment.

According to some embodiments, there is provided a composition for use in treatment of hemorrhoids in a subject in need thereof, the composition comprises or includes about 12% by weight of CBD oil, about 17% by weight of Jasminum Flower extract, about 30% by weight of Pistacia lentiscus extract, about 10% by weight pomegranate peel powder, about 7% by weight of witch hazel extract; about 10% by weight of Calendula flower extract, about 7% by weight of Achillea millefolium extract, and about 7% by weight of Ruscus aculeatus root extract. Each possibility is a separate embodiment.

According to some embodiments, the composition is administered 1, 2, 3, 4, 5 or 6 times a day.

According to some embodiments, the composition is in the form of suppositories. According to some embodiments, each suppository comprises from 10 to 400 mg CBD. According to some embodiments, each suppository comprises from 10 to 350, from 20 to 300, from 35 to 250, from 40 to 200, from 45 to 150, from 50 to 100 mg CBD. According to one embodiment, each suppository comprises from 40 to 120 or from 50 to 110 mg of CBD. According to some embodiments, the composition is administered 1, 2, 3, 4, 5 or 6 times a day. According to one embodiment, the suppository comprises from 50 to 110 of from 60 to 110 mg of CBD and is administered 2 or 3 times a day.

According to some embodiment, the composition is co-administered with an active agent being a standard treatment of the disorder. According to one embodiment, the active agent is administered in a concentration or in a frequency lower than in the standard treatment. The term “co-administration” encompasses administration of a first and second agent in an essentially simultaneous manner, such as in a single dosage form, e.g., a capsule or tablet having a fixed ratio of first and second amounts, or in multiple dosage forms for each. The agents can be administered in a sequential manner in either order. When co-administration involves the separate administration of each agent, the agents are administered sufficiently close in time to have the desired effect (e.g., complex formation). The term “sequential manner” refers to an administration of two compounds at different times, and optionally in different modes of administration. The agents can be administered in a sequential manner in either order. The terms “substantially simultaneous manner” refers to administration of two compounds with only a short time interval between them. In some embodiments, the time interval is in the range of from 0.5 to 60 minutes.

According to some embodiments, there is provided a method for treating anorectal disease, such as hemorrhoids. According to certain embodiments, the method provides treating an anorectal disease in a subject in need thereof, comprising administering to the subject the composition of the invention. According to one embodiment, the anal condition is hemorrhoids. According to some embodiments, there is provided a method for treating hemorrhoids in a subject in need thereof, comprising administering to the subject the composition of the invention. According to some embodiments, the method comprises administering the composition, e.g. a pharmaceutical composition of the present invention. According to some embodiments, the composition comprises from about 5 to about 30% by weight of CBD oil, from about 15 to about 30% by weight of Jasminum Flower extract, from about 15 to about 50% by weight of Pistacia lentiscus extract, from about 5 to about 25% by weight of pomegranate peel powder, from about 2.5 to about 15% by weight of witch hazel extract, from about 2.5 to about 25% by weight of Calendula flower extract, from about 2.5 to about 25% by weight of Achillea millefolium extract, from about 2.5 to about 25% by weight of Ruscus aculeatus root extract.

In some embodiments, the composition or the pharmaceutical composition of the invention is formulated for local administration. In some embodiments, the composition or the pharmaceutical composition of the invention is formulated for topical administration. Formulations for topical administration include ointments, oily solution, gels, creams, lotions, solution, balm and suppository. Exemplary suitable carriers include vegetable or mineral oils, white petrolatum, branched chain fats or oils, animal fats and waxes. Stabilizers, humectants, preservatives and antioxidants may also be included, as well as agents imparting color or fragrance, if desired.

In some embodiments, the compositions may conveniently be presented in unit dosage form, and may be prepared by any of the methods well-known in the art. The selected dosage form (for example, gel, ointment, cream, suppository, etc.), may depend upon the severity of the hemorrhoids, type of hemorrhoids (internal/external) and/or the duration of the treatment.

In some embodiments, the administration regime may be determined while taking into account the clinical condition of the subject, the affected severity of the hemorrhoids, type of hemorrhoids, the scheduling of administration, the subject's age, gender, body weight and other factors relevant to clinicians of ordinary skill in the art.

In some embodiments, the compositions of the invention may be provided in a pack, suppository, or dispenser device, such as a kit. In addition, the kit may include one or more carrier(s) and/or excipients.

In some embodiments, the kit may include the composition of the invention and optionally one or more suitable excipients or carriers and instructions for use of the kit in the treatment of hemorrhoids.

According to another aspect, the present invention provides a composition comprising from about 5 to about 30% by weight of CBD oil, from about 15 to about 30% by weight of Jasminum Flower extract, from about 15 to about 50% by weight of Pistacia lentiscus extract, from about 5 to about 25% by weight of pomegranate peel powder, from about 2.5 to about 15% by weight of witch hazel extract, from about 2.5 to about 25% by weight of Calendula flower extract, from about 2.5 to about 25% by weight of Achillea millefolium extract, from about 2.5 to about 25% by weight of Ruscus aculeatus root extract for preparation of a medicament for treating an anorectal disorder such as hemorrhoids.

As used herein, the singular form “a,” “an,” and “the” include plural references unless the context clearly dictates otherwise. For example, the term “a compound” or “at least one compound” may include a plurality of compounds, including mixtures thereof.

The term “comprising” means that other steps and ingredients that do not affect the final result can be added. This term encompasses the terms “consisting of” and “consisting essentially of”.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention.

### EXAMPLES

### Example 1. Plant Extraction Process

The following extraction process was employed to obtain the following oil plant extracts:

1. Plant material was ground in the presence of ethanol to form a mixture of ground plant material and ethanol.

2. The resulting mixture was incubated for several days, depending on the type of plant and ambient temperature.

3. After incubation, the mixture was filtered.

4. After the filtration, ethanol was evaporated to receive concentrated extract.

5. For cannabis extract only: the concentrated extract was heated in a vacuum oven in order to activate the molecules.

The homogenous obtained extracts, including CBD oil, were used for the preparation of the tested compositions. An exemplary CBD oil obtained from a particular cannabis species comprises about 61.5 wt % of CBD (plus about 2 wt % CBDA). Additional components that were detected in the CBD oil were THC (about 2.5 wt %), and cannabichromene (about 1.8 wt %). It is clear that the content may vary due to different extraction conditions and cannabis strains.

### Example 2. Compositions (Formulations) for Treatment of Hemorrhoids

Formulations for topical application were prepared as described below, and tested on ameliorating hemorrhoids (pain and/or swelling).

Formulation #1 comprised a distilled pomegranate peel liquor with Pistacia lentiscus extract. The obtained formulation was not homogenous. The formulation was applied by hand at the anus of treated subjects suffering from hemorrhoids. The treated subjects reported only a mild relief.

Formulation #2 included an extract of Jasminum flower extract, Pistacia lentiscus extract, pomegranate peel powder and Calendula flower extract at equal amounts (0.25 gr of each, 1 gr in total, i.e. 25 wt % of each component). The composition was tested on 8 patients. After application of the formulation at the anus of treated subjects suffering from hemorrhoids, the subjects reported improved pain relief. Nevertheless, the results still were not satisfying.

Based on Formulation 2 several additional formulations were prepared and tested. As a first step, 5 wt % of CBD oil were added (Formulation 2A). The same 8 subjects that tested Formulation and one additional subjects were engaged in this and further experiments. In some particular examples, CBD oil comprised about 61 wt % of CBD. The CBD oil contained also additional cannabinoids such as THC (about 2.5 wt %). Upon addition of 5 wt % CBD, the patients reported a substantial improvement. The concentration of CBD oil was increased to 10 wt %, and after that to 15 wt % (Formulations 2B and 2C, respectively). While using compositions comprising 10 wt % all patients reported improvement in comparison to compositions with 5 wt % CBD, using compositions with 15 wt % some of the subject started experiencing minor adverse effects. In an additional set of trials, it was established that the most promising concentration of CBD oil was about 12 wt %, although some of the patients preferred higher CBD oil concentrations. The formulation (Formulation 2D) that provided the best effect at that stage comprises 12 wt % of CBD oil, and 22 wt % of each of: Jasminum flower extract, Pistacia lentiscus extract, pomegranate peel powder and Calendula flower extract.

At the next stage, we added 3 additional extracts: extracts of Calendula flower extract, Achillea millefolium extract, and Ruscus aculeatus root extract in equal amounts to provide Formulation #3.

Formulation #3, which provided the most favorable results included 12% (weight) of CBD oil, 17% by weight of Jasminum flower extract, 30% by weight of Pistacia lentiscus extract, 10% by weight pomegranate peel powder, 7% by weight of witch hazel extract; 10% by weight of Calendula flower extract, 7% by weight of Achillea millefolium extract, and 7% by weight of Ruscus aculeatus root extract.

After application of the formulation 3 at the anus of treated subjects suffering from hemorrhoids (twice daily), 100% of the subjects reported a substantial pain relief, up to complete pain elimination, and over 60% of subjects reported reduced swelling, constriction of blood vessels and their internalization into the anus. Formulation #3 was much more efficient than formulation 2D.

The results obtained with Formulation (composition) #3 clearly demonstrate an enhanced therapeutic effect on hemorrhoids, by reducing or eliminating pain as well as reducing swelling and constriction of blood vessels.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-25. (canceled)

26. A composition comprising from about 5 to about 30% by weight of CBD oil, from about 10 to about 30% by weight of Jasminum flower extract, from about 15 to about 50% by weight of Pistacia lentiscus extract, from about 5 to about 25% by weight pomegranate peel powder, from about 2.5 to about 15% by weight of witch hazel extract, from about 2.5 to about 25% by weight Calendula flower extract, from about 2.5 to about 25% by weight Achillea millefolium extract, and from about 2.5 to about 15% by weight Ruscus aculeatus root extract.

27. The composition according to claim 26, comprising from about 10 to about 20% by weight of CBD oil.

28. The composition according to claim 26, wherein said CBD oil comprises from about 55 to about 80% by weight of CBD.

29. The composition according to claim 26, wherein the CBD oil is obtained from plant extract, or is synthetically prepared.

30. The composition according to claim 26, comprising from about 12 to about 22% by weight of Jasminum flower extract.

31. The composition according to claim 26, comprising from about 25 to about 35% by weight of Pistacia lentiscus extract.

32. The composition according to claim 26, comprising from about 5 to about 15% by weight of pomegranate peel powder.

33. The composition according to claim 26, comprising from about 2.5 to about 10% witch hazel extract.

34. The composition according to claim 26, comprising from about 5 to about 15% Calendula flower extract.

35. The composition according to claim 26, comprising from about 2.5 to about 10% Achillea millefolium extract.

36. The composition according to claim 26, comprising from about 2.5 to about 10% Ruscus aculeatus root extract.

37. The composition according to claim 26, comprising from about 8 to about 22% by weight of CBD oil, from about 12 to about 22% by weight of Jasminum Flower extract, from about 25 to about 35% by weight of Pistacia lentiscus extract, from about 5 to about 15% by weight pomegranate peel powder, from about 2.5 to about 12% by weight of witch hazel extract, from about 7 to about 12% by weight Calendula flower extract, from about 5 to about 10%, by weight Achillea millefolium extract, and from about 5 to about 10% by weight Ruscus aculeatus root extract.

38. The composition according to claim 37, wherein said CBD oil comprises from about 55 to about 80% by weight of CBD.

39. The composition according to claim 26, further comprising a topically acceptable carrier.

40. The composition according to claim 26, wherein the composition is a pharmaceutical composition comprising one or more pharmaceutically acceptable excipients.

41. The composition according to claim 26, wherein the composition is (i) formulated for a local administration; (ii) in the form of a paste, a gel, a cream, an ointment, an oily solution, or a suppository; or (iii) both (i) and (ii).

42. The composition according to claim 41, wherein the local administration is topical.

43. A method for treating an anorectal disorder in a subject in need thereof comprising administering to the subject a composition comprising from about 5 to about 30% by weight of CBD oil, from about 15 to about 30% by weight of Jasminum flower extract, from about 15 to about 50% by weight of Pistacia lentiscus extract, from about 5 to about 25% by weight pomegranate peel powder, from about 2.5 to about 15% by weight of witch hazel extract, from about 2.5 to about 25% by weight Calendula flower extract, from about 2.5 to about 25% by weight Achillea millefolium extract, and from about 2.5 to about 15% by weight Ruscus aculeatus root extract.

44. The method according to claim 43, wherein (i) the anorectal disorder is hemorrhoids; (ii) the composition is administered topically; or (iii) both (i) and (ii).

45. The method according to claim 44, wherein the hemorrhoids are internal or external.
